(19) United States
(12) Patent Application Publication
(10) Pub. No.: US 2007/0179496 A1
(43) Pub. Date: Aug. 2, 2007

(54) Flexible catheter for ablation therapy

(75) Inventors: John M. Swoyer, Andover, MN (US); Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/343,967

(22) Filed: Jan. 31, 2006

(51) Int. Cl. A61B 18/14

(52) U.S. Cl. 606/41

(57) ABSTRACT

The disclosure describes a system that may be used to deliver ablation therapy to a target tissue within a patient. The system uses a flexible catheter to navigate through a passage and reach a target tissue. Once at the desired location, a side of the flexible catheter is forced against a wall of the passage to allow a needle to be extended from the side of the flexible catheter and into the target tissue. A pull-wire or inflatable balloon may be used as the control mechanism that forces the flexible catheter against the passage wall. In the case of the pull-wire, the flexible catheter may also be steered through the passage. As an example, the flexible catheter may be inserted into the urethra and the needle may be deployed into the prostate to treat benign prostatic hypertrophy (BPH).

### TECHNICAL FIELD

The invention relates to medical devices and, more particularly, to devices for delivering therapy to a tissue.

### BACKGROUND

Tissue ablation is a commonly used surgical technique to treat a variety of medical conditions. Medical conditions may include excess tissue growth (such as benign prostatic hypertrophy), benign tumors, malignant tumors, destructive cardiac conductive pathways (such as ventricular tachycardia), and even sealing blood vessels during surgical procedures. Treatment for these medical conditions may include removing or destroying the target tissue, of which ablation is an appropriate solution.

Typically, ablation therapy involves heating the target tissue with a surgical instrument such as a needle or probe. The needle is coupled to an energy source which heats the needle, the target tissue, or both. Energy sources may cause ablation through radio frequency (RF) energy, heated fluids, impedance heating, or any combination of these sources. The needle may be presented to the target tissue during an open surgical procedure or through a minimally invasive surgical procedure.

As an example, benign prostatic hypertrophy (BPH) is a condition caused by the second period of continued prostate gland growth. This growth begins after a man is approximately 25 years old and may begin to cause health problems after 40 years of age. The prostate growth eventually begins to constrict the urethra and may cause problems with urination and bladder functionality. Minimally invasive ablation therapy may be used to treat this condition. A catheter is inserted into the urethra of a patient and directed to the area of the urethra adjacent to the prostate. An ablation needle is extended from the catheter and into the prostate. The clinician performing the procedure selects the desired ablation parameters and the needle heats the prostatic tissue. Ablation therapy shrinks the prostate to a smaller size that no longer interferes with normal urination and bladder functionality, and the patient may be relived of most problems related to BPH.

### SUMMARY

The disclosure describes a system that may be used to deliver ablation therapy to a target tissue within a patient. Since tissue ablation may be performed through minimally invasive surgical procedures, a device needs to navigate internal passages to reach the target tissue. Utilizing a flexible catheter may reduce the difficulty of navigating these internal passages and also reduce any pain experienced by the patient during the procedure.

Additionally, the flexible catheter may further facilitate placement through the use of a control mechanism. The control mechanism may be automatically operated by the system or manually operated by a clinician. Once at the desired location, a side of the flexible catheter is forced against a wall of the passage to allow one or more needles to be extended from the flexible catheter and into the target tissue. For example, a pull-wire, an inflatable balloon or the like may be used as the control mechanism that forces the flexible catheter against the passage wall. In the case of the pull-wire, the flexible catheter may also be steered through the passage. As an example, the flexible catheter may be inserted into the urethra and the needle may be deployed into the prostate to treat benign prostatic hypertrophy (BPH).

In one embodiment, this disclosure is directed to a method for ablating tissue that includes inserting a flexible catheter into a passage, forcing a first side of the flexible catheter against a wall of the passage, extending a needle from the first side of the flexible catheter through the wall of the passage and into a target tissue, and delivering energy via a needle to ablate at least a portion of the target tissue.

In another embodiment, this disclosure is directed to a system for ablating tissue that includes a generator that generates energy to ablate at least a portion of a target tissue, a flexible catheter that is inserted into a passage, a housing that accepts the flexible catheter, a control mechanism that forces a first side of the flexible catheter against a wall of the passage, and a needle that extends from the first side of the flexible catheter through the wall of the passage and into the target tissue to deliver the energy.

In an additional embodiment, this disclosure is directed to a device for accessing a tissue to be ablated that includes a flexible catheter that is inserted into a passage, a control mechanism that forces a first side of the flexible catheter against a wall of the passage, a needle that extends from the first side of the flexible catheter through the wall of the passage and into a target tissue, wherein the first side of the flexible catheter is near a distal end of the flexible catheter, and an axial channel that accepts a flexible cystoscope.

In various embodiments, the device described in this disclosure may provide one or more advantages. Inserting a flexible catheter into a patient may be easier for a clinician and less painful for a patient than inserting a rigid catheter. Moreover, a control mechanism may reduce any separation of tissue from the flexible catheter tip when a needle punctures the adjacent tissue. The control mechanism may also cause the depth of needle penetration in the target tissue to be more predictable. In addition, the flexible catheter may be capable of navigating an increased variety of body passages. Other advantages may include not having to insulate a metallic rigid catheter and incorporating a flexible cystoscope.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a conceptual diagram illustrating an example generator system in conjunction with a patient.

FIG. 2 is a side view of an example hand piece and connected catheter that delivers therapy to target tissue.

FIGS. 3A, 3B and 3C are side views of exemplary flexible catheters which may bend at different locations and to different degrees.

FIG. 4 is a cross-sectional side view of a flexible catheter inserted into a urethra of a patient.

FIGS. 5A, 5B and 5C are cross-sectional side views of a distal end of exemplary flexible catheters in which a pull-wire is used as a control mechanism.

FIGS. 6A and 6B are exemplary hand pieces that provide control of a pull-wire.

FIGS. 7A, 7B and 7C are cross-sectional side views of a distal end of and exemplary flexible catheter in which an inflatable balloon is used as a control mechanism.

FIGS. 8A and 8B are exemplary hand pieces that provide control of an inflatable balloon.

FIG. 9 is functional block diagram illustrating components of an exemplary generator system.

FIG. 10 is functional block diagram illustrating components of an exemplary generator system that controls an inflatable balloon.

FIG. 11 is a flow diagram illustrating an example technique for positioning a flexible catheter with a pull-wire and ablating tissue.

FIG. 12 is a flow diagram illustrating an example technique for positioning a flexible catheter with an inflatable balloon and ablating tissue.

### DETAILED DESCRIPTION

In one aspect, this disclosure is directed to a flexible catheter used in an ablation system that provides ablation therapy within a patient. Tissue ablation may be performed in an open surgical procedure or in a minimally invasive procedure. During a minimally invasive procedure, the flexible catheter is inserted into a patient until it reaches a target tissue that is to be ablated.

The flexible catheter includes a control mechanism to force a side of the catheter against a passage or tissue such that a needle may be deployed into the adjacent tissue. The flexible catheter may allow easier catheter insertion and reduce patient pain without sacrificing therapy efficacy. The control mechanism forces a side of the flexible catheter against the adjacent tissue to facilitate deploying at least one needle into the target tissue. The needle heats the target tissue to a temperature that causes tissue ablation. In addition to providing a bias against the adjacent tissue, a pull-wire may enable a clinician to steer the catheter within a passage of the patient to reach the ablation site.

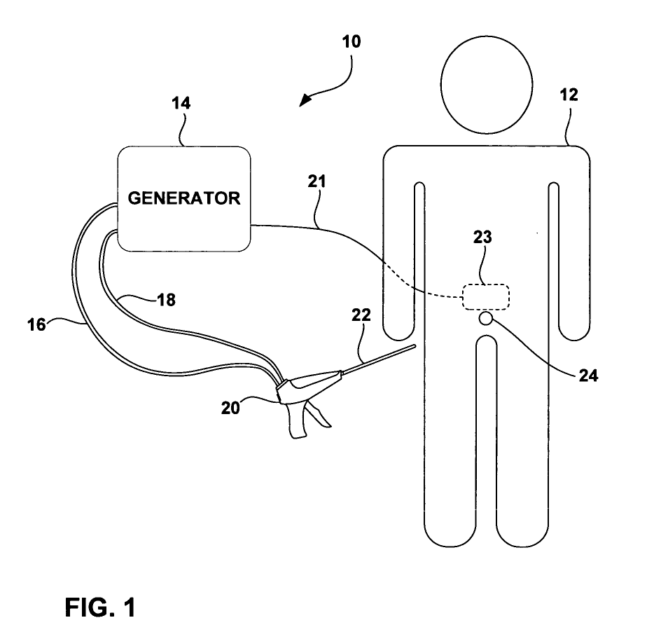

FIG. 1 is a conceptual diagram illustrating an example generator system in conjunction with a patient. As shown in the example of FIG. 1, system 10 may include a generator 14 that delivers therapy to treat a condition of patient 12. In this exemplary embodiment, generator 14 is a radio frequency (RF) generator that provides RF energy to heat tissue of the prostate gland 24. This ablation of prostate tissue destroys a portion of the enlarged prostate caused by, for example, benign prostatic hyperplasia (BPH). The RF energy is transmitted through electrical cable 16 to therapy device 20. The energy is then transmitted through a flexible catheter 22 and is delivered to prostate 24 by a needle electrode (not shown in FIG. 1). In addition to the needle, a conductive fluid may be pumped out of delivery device 14, through tubing 18, into therapy device 20, and through flexible catheter 22 to interact with the RF energy being delivered by the needle. This “wet electrode” may increase the effective heating area of the needle and increase therapy efficacy. Ground pad 23 is placed at the lower back of patient 12 and connected to generator 14 by wire 21 to return the energy emitted by the needle electrode.

In the illustrated example, generator 14 is an RF generator that includes circuitry for developing RF energy from an included rechargeable battery or a common electrical outlet. The RF energy is produced within parameters that are adjusted to provide appropriate prostate tissue heating. The RF current is conveyed from generator 14 via electrical cable 16 which is connected to the generator. The conductive fluid is provided to the needle by a pump (not shown) also located within generator 14. In some embodiments, a conductive fluid may not be used in conjunction with the RF energy. This embodiment may be referred to as a “dry electrode” ablation system. Alternatively, other energy sources may be used in place of RF energy.

Therapy energy and other associated functions such as fluid flow may be controlled via a graphic user interface located on a color liquid crystal display (LCD), or equivalent screen of generator 14. The screen may provide images created by the therapy software, and the user may interact with the software by touching the screen at certain locations indicated by the user interface. In this embodiment, no additional devices, such as a keyboard or pointer device, are needed to interact with the device. The touch screen may also enable device operation. In some embodiments, the device may require an access code or biometric authorization to use the device. Requiring the clinician to provide a fingerprint, for example, may limit unauthorized use of the system. Other embodiments of generator 14 may require input devices for control, or the generator may require manual operation and minimal computer control of the ablation therapy.

Connected to generator 14 are a cable 16 and a tube 18. Cable 16 conveys RF energy and tube 18 conducts fluid from generator 14 to therapy device 20. Tube 18 may carry conductive fluid and cooling fluid to the target tissue, or an additional tube (not shown) may carry the cooling fluid used to irrigate the urethra of patient 12. Therapy device 20 may be embodied as a hand-held device as shown in FIG. 1. Therapy device 20 may include a trigger to control the start and stop of therapy. The trigger may be pressure sensitive, where increased pressure of the trigger provides an increased amount of RF energy or increase the fluid flow to the tissue of prostate 24. The trigger may also deploy the needle into the target tissue. Attached to the distal end of therapy device 20 is a flexible catheter 22. Flexible catheter 22 may provide a conduit for both the RF energy and the fluid. System 10 may utilize one, two, or three or more needles to deliver RF energy into prostate 24.

Additionally, flexible catheter 22 may include a control mechanism (not shown) for positioning the catheter and/or the needle. The control mechanism may vary widely depending on the shape of the flexible catheter 22 and the intended surgical procedure to be performed, but suitable examples include a pull-wire located within flexible catheter 22 or an inflatable balloon located on the catheter opposite an exit opening of the needle. Therefore, in addition to facilitating positioning of the catheter 22, the control mechanism may facilitate needle penetration into the tissue. For example, in a BPH treatment protocol, since flexible catheter 22 would be entering patient 12 through the urethra, the catheter may be very thin in diameter and long enough to reach the prostate in most any anatomical dimensions.

The end of flexible catheter 22 may contain one or more electrodes for delivering RF current to the tissue of enlarged prostate 24. Flexible catheter 22 may contain one or more needles that are each an electrode for penetrating into an area of prostate 24 from the urethra. When RF energy is being delivered, the target tissue increases in temperature, which destroys a certain volume of tissue. This heating may last a few seconds or a few minutes, depending on the condition of patient 12. In some embodiments, a conductive fluid may exit small holes in the needle and flow around the electrode. This conducting fluid, e.g., saline, may increase the effective heating area and decrease the heating time for effective treatment. Additionally, ablating tissue in this manner may enable the clinician to complete therapy by repositioning the needles a reduced number of times.

In some cases, therapy device 20 may only be used for one patient. Reuse may cause infection and contamination, so it may be desirable for therapy device 20 to only be used once. A feature on therapy device 20 may be a smart chip in communication with generator 14. For example, when the therapy device is connected to generator 14, the generator may request use information from the therapy device. If the device has been used before, generator 14 may disable all functions of the therapy device to prevent reuse of the device. Once therapy device 20 has been used, the smart chip may create a use a log to identify the therapy delivered and record that the device has been used. The log may include graphs of RF energy delivered to the patient, total RF energy delivered in terms of joules or time duration, error messages created, measure tissue properties, end lesion volume, or any other pertinent information to the therapy.

In other embodiments, different catheters 22 may include different configurations of the needle electrode, such as lengths, diameters, number of needles, or sensors in the needle that detect temperature or other tissue properties. In this manner, a clinician may select the desired catheter 22 that provides the most efficacious therapy to patient 12.

While the example of system 10 described herein is directed toward treating BPH in prostate 24, system 10 may utilize a flexible catheter and control mechanism for ablation feedback at any other target tissue of patient 12. For example, the target tissue may be polyps in a colon, a kidney tumor, esophageal cancer, uterine cancer tissue, or liver tumors. In any case, a flexible catheter 22 may reduce the difficulty of positioning the catheter within patient 12 which may reduce the pain experienced by the patient during the procedure.

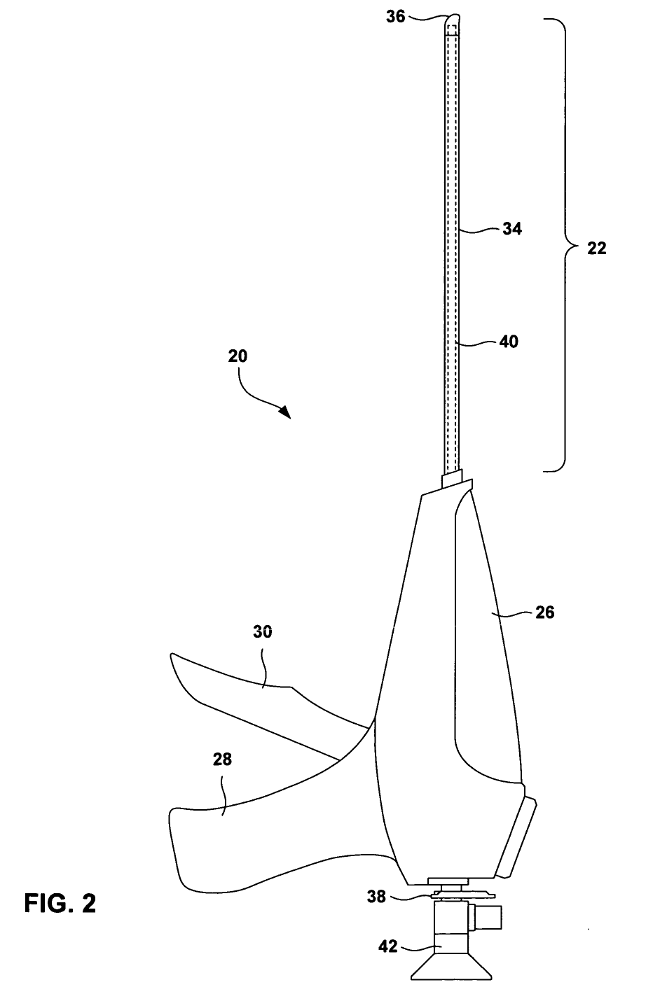

FIG. 2 is a side view of an example hand piece and connected catheter that delivers therapy to target tissue. As shown in FIG. 2, therapy device 20 includes housing 26 which is attached to handle 28 and trigger 30. Cystoscope 40 may be inserted though an axial channel in housing 26, locked with lock 38, and fitted within flexible catheter 22. Flexible catheter 22 includes shaft 34 and tip 36. A clinician holds handle 28 and trigger 30 to guide flexible catheter 22 through a urethra. The clinician looks though eyepiece 42 of cystoscope 40 to view the urethra through tip 36 and locate a prostate, or other appropriate anatomical land marks, for positioning flexible catheter 22 adjacent to prostate 24. Once the clinician identifies correct placement for tip 36, trigger 30 is squeezed toward handle 28 to extend the needle into prostate 24.

Housing 26, handle 28 and trigger 30 are constructed of a lightweight molded plastic such as polystyrene. In other embodiments, other injection molded plastics may be used such as polyurethane, polypropylene, high molecular weight polyurethane, polycarbonate or nylon. Alternatively, construction materials may be aluminum, stainless steel, a metal alloy or a composite material. In addition, housing 26, handle 28 and trigger 30 may be constructed of different materials instead of being constructed out of the same material. In some embodiments, housing 26, handle 28 and trigger 30 may be assembled through snap fit connections, adhesives or mechanical fixation devices such as pins or screws.

Shaft 34 of flexible catheter 22 may be fixed into a channel of housing 26 or locked in place for a treatment session. Flexible catheter 22 may be produced in different lengths or diameters with different configurations of needles or tip 36. A clinician may be able interchange flexible catheter 22 with housing 26. In other embodiments, flexible catheter 22 may be manufactured within housing 26 such that the clinician may have to use therapy device 20 as one medical device.

Shaft 34 may be completely flexible or contain a section that is rigid. The flexible portion may be constructed of a polymer such as nylon or polyurethane with or without a braided reinforcing material constructed of stainless steel or another polymer. The rigid portion may be manufactured of stainless steel or another metal alloy and insulated with a polymer such as nylon or polyurethane. Alternatively, the rigid portion of shaft 34 may be constructed of a rigid polymer or composite material. Shaft 34 includes one or more channels that house one or more needles, a cystoscope, and the control mechanism. Tip 36 is constructed of an optically clear polymer such that the clinician may view the urethra during flexible catheter 22 insertion. Shaft 34 and tip 36 may be attached with a screw mechanism, snap fit, or adhesives. Tip 36 also includes openings that allow the first and second needles to exit catheter 22 and extend into prostate 24. At least a portion of flexible catheter 22 may be lubricated to facilitate introduction into urethra 51. Alternatively, shaft 34 may be hydrophilic or lubricious when wet to aid insertion. Flexible catheter 22 may be sterilizable or disposable to reduce contamination between different patients. In addition, the entire therapy device 20 may be sterilizable or disposable.

Cystoscope 40 may be rigid or flexible. If a rigid cystoscope is inserted into flexible catheter 22, the flexible catheter may become rigid for use. Using a rigid cystoscope may also add stress to the cystoscope and potentially cause damage to the cystoscope. A flexible cystoscope may allow full or partial flexible catheter 22 movement. In some cases, cystoscope 40 may need to be removed in order for flexible catheter 22 to be fully functional. Alternatively, a small camera may be located in tip 36 with a fiber optic or cable running through the axial channel of flexible catheter 22.

In some embodiments, housing 26, handle 28, or trigger 30 may include dials or switches to control the deployment of the one or more needles in unison or independently. For example, multiple first needles may be employed to treat a larger volume of tissue at one time. In other embodiments, inserting cystoscope 40 may disable one or more features of flexible catheter 22, such as certain control mechanisms.

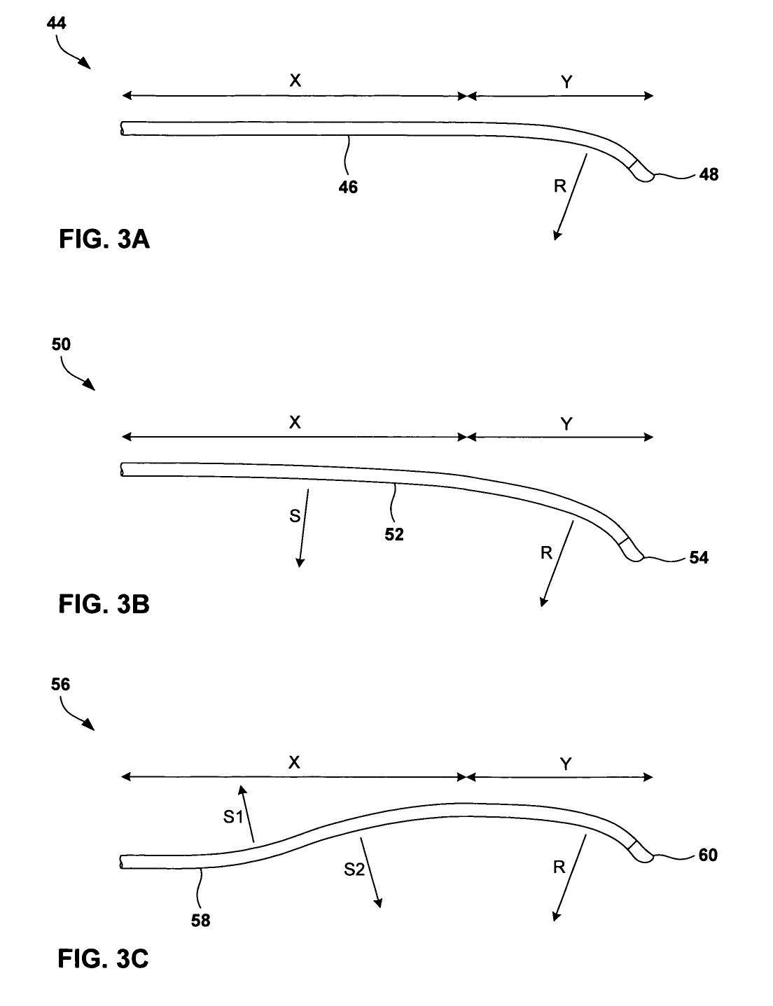

FIGS. 3A, 3B and 3C are side views of exemplary flexible catheters which may bend at different locations and to different degrees. Similar to FIG. 2, shafts 34, 46, 52 or 58 may be constructed of one or more materials. Any flexible or semi-rigid portions of shafts 34, 46, 52 or 58 may be constructed of a polymer such as nylon or polyurethane. A braided material constructed of stainless steel or a stiff polymer may be included in the polymer to provide support resisting buckling. In addition, flexible or semi-rigid portions of shafts 34, 46, 52 or 58 may be constructed from a spiraled cut or sectioned stainless steel tube of which thin areas of the tube allow a degree of pliability. The stainless tube may be coated with a polymer for insulation and biocompatibility. The rigid portion may be manufactured of stainless steel or another metal alloy and insulated with a polymer such as nylon or polyurethane. Alternatively, the rigid portion of shafts 34, 46, or 52 may be constructed of a rigid polymer or composite material.

As shown in FIG. 3A, flexible catheter 44 is an embodiment of flexible catheter 22 and includes a rigid portion. Flexible catheter 44 includes shaft 46 and tip 48 which are exemplary embodiments of the shaft 34 and the tip 36, respectively, shown in FIG. 2. Tip 48 is identical to tip 36 of FIG. 2. Shaft 46 includes a rigid portion and a flexible portion. Length X of shaft 46 is the rigid portion of the shaft, where length X is between approximately 10 and 90 percent of the length of shaft 46. Length Y of shaft 46 is the flexible portion of the shaft, where length Y is between approximately 10 and 90 percent of the total length of shaft 46. Radius of curvature R of length Y is sufficient to bend through most anatomical passages. Generally, radius of curvature R is greater than 2 cm. The stiffness of length X of shaft 46 may provide support so that a clinician may guide flexible catheter 44 through the urethra. In some embodiments, flexible catheter 44 may include multiple rigid portions that may or may not be separated by flexible portions similar to length Y.

As shown in FIG. 3B, flexible catheter 50 is an embodiment of flexible catheter 22 and includes a semi-rigid portion. Flexible catheter 50 includes shaft 52 and tip 54 which are embodiments of shaft 34 and tip 36, respectively, of FIG. 2. Tip 54 is identical to tip 36 of FIG. 2. Shaft 52 includes a semi-rigid portion and a flexible portion. Length X of shaft 52 is the semi-rigid portion of the shaft, where length X is between approximately 10 and 90 percent of the length of shaft 52. Radius of curvature S is minimal to provide support to flexible catheter 50 while allowing the flexible catheter to bend slightly. Therefore, radius of curvature S is generally greater than 20 cm. Length Y of shaft 52 is the flexible portion of the shaft, where length Y is between approximately 10 and 90 percent of the total length of shaft 52. Radius of curvature R of length Y is sufficient to bend through most anatomical passages. Generally, radius of curvature R is greater than 2 cm. In some embodiments, flexible catheter 50 may include multiple semi-rigid portions that may or may not be separated by flexible portions similar to length Y. Both semi-flexible length X and flexible length Y may bend in any direction, at any location along shaft 52.

FIG. 3C shown flexible catheter 56 as an embodiment of flexible catheter 22. Flexible catheter 56 is completely flexible throughout the length of the catheter. Flexible catheter 56 includes shaft 58 and tip 60 which are embodiments of shaft 34 and tip 36, respectively, in FIG. 2. Tip 60 is identical to tip 36 of FIG. 2. Shaft 58 is completely flexible throughout lengths X and Y. Lengths X and Y are provided to identify different curvatures possible within shaft 58. Radius of curvatures S1, S2 and R are generally greater than 2 cm, such that shaft 58 may bend through most anatomical passages. While shaft 58 is shown to curve or bend in only one plane, shaft 58 may be capable of bending within a three-dimensional space.

In some embodiments, some portions of flexible catheter 58 may have slightly different properties. Length X of shaft 58 is a flexible portion of the shaft that is slightly less flexible than length Y, where length X is between approximately 10 and 90 percent of the length of shaft 58. Radius of curvature S1 and S2 may generally be greater than 6 cm. Length Y of shaft 58 is also a flexible portion of the shaft, where length Y is between approximately 10 and 90 percent of the total length of shaft 58. In other embodiments, flexible catheter 50 may include many different flexible portions within shaft 58 to facilitate insertion into the urethra or certain other body passages. Shaft 58 may be bent in any direction, at any location along shaft 58 not shown in this embodiment.

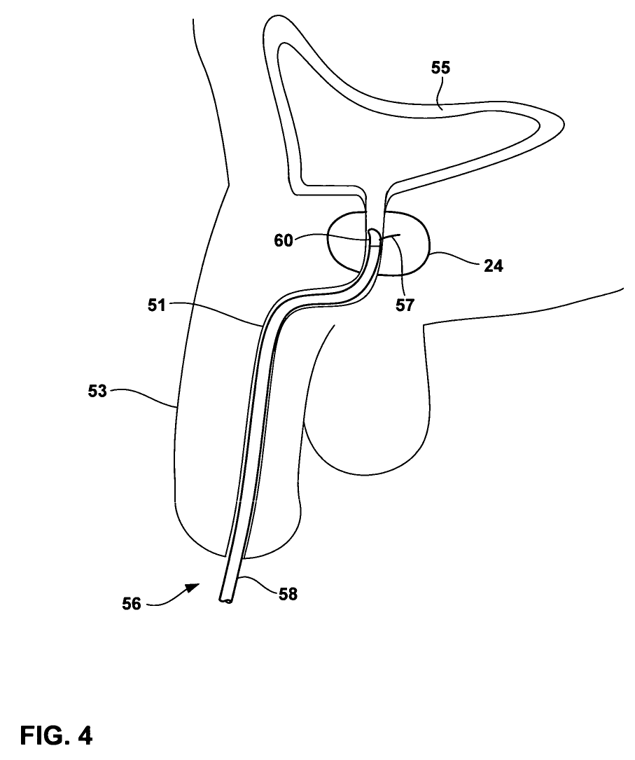

FIG. 4 is a cross-sectional side view of a flexible catheter inserted into a urethra of a patient. As shown in FIG. 4, flexible catheter 56 of FIG. 3C is inserted into urethra 51 of patient 12. Urethra 51 is within penis 53 and travels through prostate 24 before opening into bladder 55. Flexible catheter 56 includes shaft 58 and tip 60. Once flexible catheter 56 is positioned correctly, tip 60 is forced against the wall of urethra 51 adjacent to prostate 24 with a control mechanism (not shown). Needle 57 is deployed from tip 60 into prostate 24 to ablate the surrounding prostate tissue.

Flexible catheter 56 is capable of following the anatomical curves of urethra 51 to facilitate easier insertion by the clinician. Additionally, patient 12 may have less pain during the ablation procedure if flexible catheter 56 follows the natural contours of urethra 51 instead of urethra 51 and surrounding tissue conforming to the structure of a rigid catheter. Flexible catheter 56 may cause less irritation to urethra 51, which may reduce tissue swelling and the need for a drainage catheter to remain in the urethra after the procedure is completed. Additionally, flexible catheter 56 may enable the clinician to extend the catheter into bladder 55 to treat bladder tissue that a rigid catheter could not reach.

Flexible catheter 56 is used as an exemplary flexible catheter 22, and any other flexible catheters 44 or 50 could be used instead. Utilizing a rigid or partially rigid portion of flexible catheter 44 or 50 may provide structural support the flexible distal portion of each catheter when applying axial force to insert the catheter. However, a rigid or semi-rigid portion of flexible catheters 44 or 50 may require at least some length of urethra 51 to conform to the structure of each catheter.

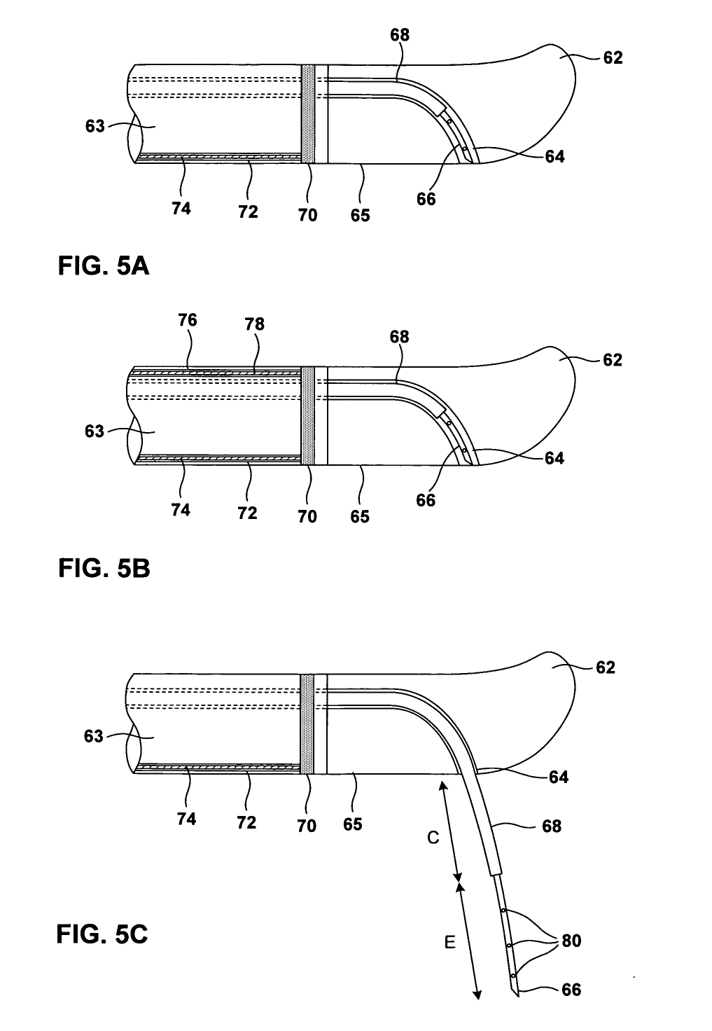

FIGS. 5A, 5B and 5C are cross-sectional side views of a distal end of exemplary flexible catheters in which a pull-wire is used as a control mechanism. FIGS. 5A, 5B and 5C may be embodiments of flexible catheters 22, 44, 50 or 56, but flexible catheter 22 is referenced herein. As shown in FIG. 5A, shaft 63 is coupled to tip 65 at the distal end of catheter 22, where shaft 63 and tip 65 are similar to shaft 34 and tip 36. Tip 65 includes protrusion 62 that aids in catheter insertion through the urethra. Tip 65 also includes channel 64 which allows needle 66 to exit tip 65. Needle 66 is insulated with sheath 68, such that the exposed portion of needle 66 may act as an electrode. A second needle (not shown) resides behind needle 66 and cannot be seen in FIG. 5A. While two needles are described herein, any number of needles may be used. Both needles may form an angle in the plane normal to the side view of FIG. 5A.

Shaft 63 also contains wire channel 72, pull-wire 74 and support ring 70 which creates a control mechanism to force the side of tip 65 against the wall of urethra 51. Support ring 70 is embedded into the distal end of shaft 63 such that it provides an anchoring point for pull-wire 74. Pull-wire 74 is attached to support ring 70 and resides within wire channel 72. Pulling pull-wire 74 towards the proximal end of flexible catheter 22 causes the catheter to deflect towards the side of the catheter containing the pull-wire. In this manner, tip 65 may be forced in once direction to deploy needle 66 or steer flexible catheter 22 within urethra 51.

Pull-wire 74 may be constructed of stainless steel, a metal alloy, or a polymer such as nylon or Kevlar. The material used to construct pull-wire 74 may have high tensile strength and tensile stiffness. Support ring 70 may also be constructed of stainless steel or another metal alloy. Polymers with high elastic modulus, such as high molecular weight polyurethane or polycarbonate, may also be used as materials for support ring 70. Pull-wire 74 may be attached to support ring 70 through adhesives, bonding (such as welding or soldering), or tying the pull-wire to the support ring. In some embodiments, pull-wire 74 may be pushed through wire channel 72 to force flexible catheter 22 to bend in the opposite position.

Channel 64 continues from tip 65 through shaft 63. The curved portion of channel 64 in tip 65 deflects needle 66 such that the needle penetrates the target tissue from the side of flexible catheter 22. The curvature of channel 64 may be altered to produce different entry angles of needle 66 and the second needle. However, the needles may not extend beyond the distal end of tip 65. In other words, the needles may exit at or near the side of flexible catheter 22, wherein the side is a lengthwise edge substantially facing the wall of urethra 51. The wall of urethra 51 is a tissue barrier as it surrounds flexible catheter 22. In some embodiments, the distal ends of needle 66 or the second needle may stop at a point further from housing 26 than the distal end of tip 65.

FIG. 5B shows an alternative embodiment of FIG. 5A, where a second pull-wire is added to flexible catheter 22. Pull-wire 78 resides within wire channel 76 and is attached to support ring 70, similar to pull-wire 74. Pull-wire 78 may allow the clinician to bend flexible catheter 22 in a direction opposite the direction of bending produced from pull-wire 74. In this manner, the multiple pull-wires may facilitate insertion of flexible catheter 22 into urethra 51. Additionally, the clinician may be able to make flexible catheter 22 rigid by pulling both pull-wires 74 and 78 simultaneously.

While pull-wires 74 and 78 are shown to be placed opposite each other, the pull-wires may be positioned at any location around the circumference of shaft 63. In this manner, flexible catheter 22 may be configured to force tip 65 to any direction. In other embodiments, more than two pull-wires may be included in shaft 63 to facilitate complex movements or shapes by flexible catheter 22.

As shown in FIG. 5C, needle 66 has been deployed from tip 65 of flexible catheter 22 after pull-wire 74 is pulled in tension. Tightening pull-wire 74 forces the side of tip 65 against urethra 51 and facilitates needle 66 penetration while reducing any space between the urethra and tip 65. The exposed length E of needle 66 is variable by controlling the position of sheath 68. The covered length C of needle 66 is that length of the first needle outside of tip 65 that is not delivering energy to the surrounding tissue. Exposed length E may be controlled by the clinician to be generally between 1 mm and 50 mm. More specifically, exposed length E may be between 12 mm and 22 mm. Covered length C may be generally between 1 mm and 50 mm. Specifically, covered length C may also be between 12 mm and 22 mm. Once needle 66 and the second needle are deployed, the needles may be locked into place until the ablation therapy is completed.

Needle 66 is a hollow needle which allows conductive fluid, i.e., saline or another fluid, to flow from generator 14 to the target tissue. Needle 66 includes multiple holes 80 which allow the conductive fluid to flow into the target tissue and increase the effective size of the needle electrode. The conductive fluid may also more evenly distribute the RF energy to the tissue to create more uniform lesions. In some embodiments, needle 66 may also include a hole at the distal tip of the needle. In other embodiments, needle 66 may only include a hole at the distal tip of the needle. Generator 14 may include a pump that delivers the conductive fluid at a predetermined flow rate, a flow rate adjusted by the clinician, or a flow rate determined automatically by sensors located in flexible catheter 22 or needle 66.

Alternatively, needle 66 may not deliver a conductive fluid to the target tissue. In this case, the needle may be solid or hollow and act as a dry electrode. Delivering energy through needle 66 without a conductive fluid may simplify the ablation procedure, but the dry electrode may require a longer ablation period to create a similarly sized lesion when compared to a wet electrode. The second needle (not shown) may deliver conductive fluid similar to needle 66 or act as a dry electrode.

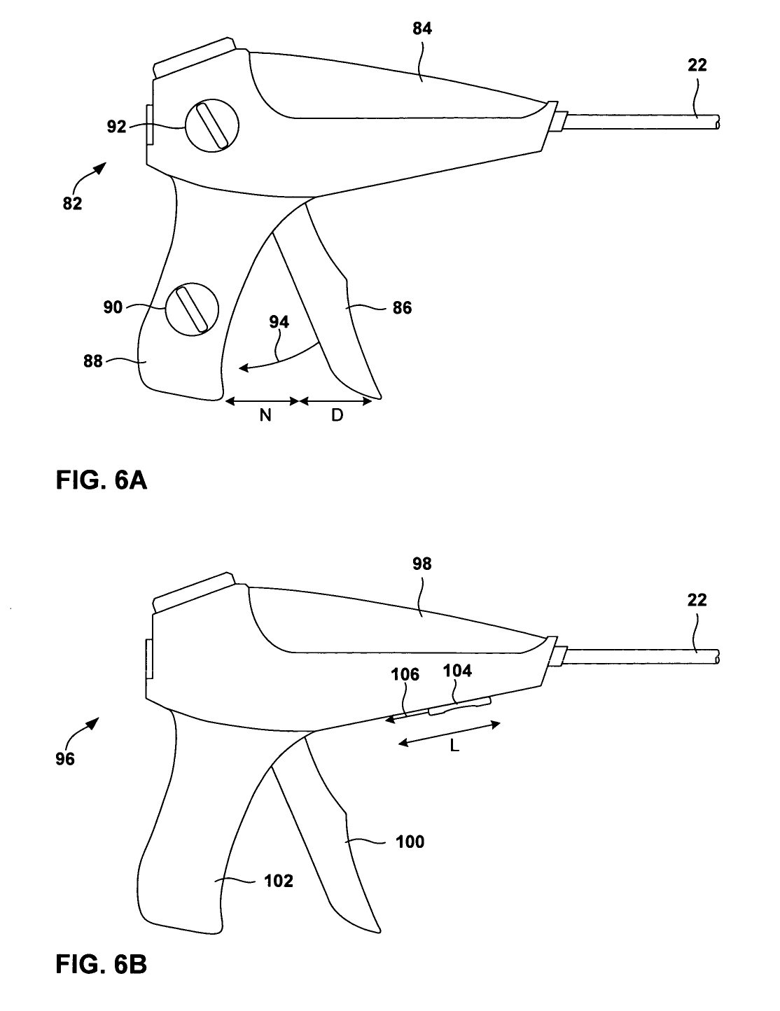

FIGS. 6A and 6B are exemplary hand pieces that provide control of a pull-wire. As shown in FIG. 6A, therapy device 82 includes housing 84, trigger 86 and handle 88 that may be alternative embodiments of housing 26, trigger 28 and handle 30 shown in FIG. 2. Therapy device 82 may be an alternative of therapy device 20 of FIG. 2. Flexible catheter 22 is inserted into housing 84, and trigger 86 and handle 88 are attached to housing 84. Dial 90 controls the length of needle 66 and any other needles while dial 92 controls the magnitude of pull-wire 74 retraction.

Before flexible catheter 22 is inserted into urethra 51, the clinician rotates dials 90 and 92 to set the limits for each mechanism. For example, dial 90 may set the deployed length of needle 66 to any length between 12 mm and 22 mm and dial 92 may limit the length pull-wire 74 can be pulled to a distance from 0 mm to 20 mm. The clinician may squeeze trigger 86 in the direction of arrow 94 towards handle 88 the distance D to bend flexible catheter 22 with pull-wire 74 and force tip 65 against the wall of urethra 51. The clinician may need to squeeze with greater force to continue moving trigger 86 the distance N. Distance N corresponds to deploying needle 66 into prostate 24. Dial 92 may include a ratcheting mechanism or locking mechanism to secure the pull-wire setting.

In some embodiments, trigger 86 may not be used to bend flexible catheter 22. Instead, dial 92 may be rotated to shorten or lengthen pull-wire 74. In this manner, dial 92 may include a ratcheting mechanism to step-wise lock the length of pull-wire 74. Alternatively, dial 92 may include a lock such that the clinician may secure the length of pull-wire 74 during ablation therapy.

In other embodiments, multiple triggers 86 or dials 92 may each operate separate pull-wires within flexible catheter 22. Alternatively, one dial may be used to selectively operate one or more pull-wires. Some embodiments may include dials 90 and 92 located on any side of housing 84 or handle 88, or in different locations on therapy device 82.

In other alternative embodiments, pull-wire 74 may be operated with a spring loaded release mechanism. Dial 92 may be used to determine the amount of force, or preload, to apply to pull-wire 74. A button may be pressed that releases the spring in the release mechanism to tighten pull-wire 74 to the predetermined force. In this manner, a clinician may not over tighten pull-wire 74 and compromise the integrity of urethra 51 or surrounding tissue.

FIG. 6B, shows therapy device 96 including housing 98, trigger 100 and handle 102 that may be alternative embodiments of housing 26, trigger 28 and handle 30 of FIG. 2. Therapy device 96 may be an alternative of therapy device 20 of FIG. 2. Flexible catheter 22 is inserted into housing 98, and trigger 100 and handle 102 are attached to housing 98. Slide 104 controls the length of pull-wire 74. Slide 104 may be moved in the direction of arrow 106 a distance L to tighten pull-wire 74 and bend flexible catheter 22. Slide 104 may include a ratcheting mechanism or lock to secure the position of pull-wire 74.

In some embodiments, slide 104 may be located on another location of therapy device 96. For example, slide 104 may be located on top of housing 98, on trigger 100 or at the side of handle 102. In other embodiments, multiple slides 104 may each operate separate pull-wires within flexible catheter 22. Alternatively, one slide may be used to selectively operate one or more pull-wires.

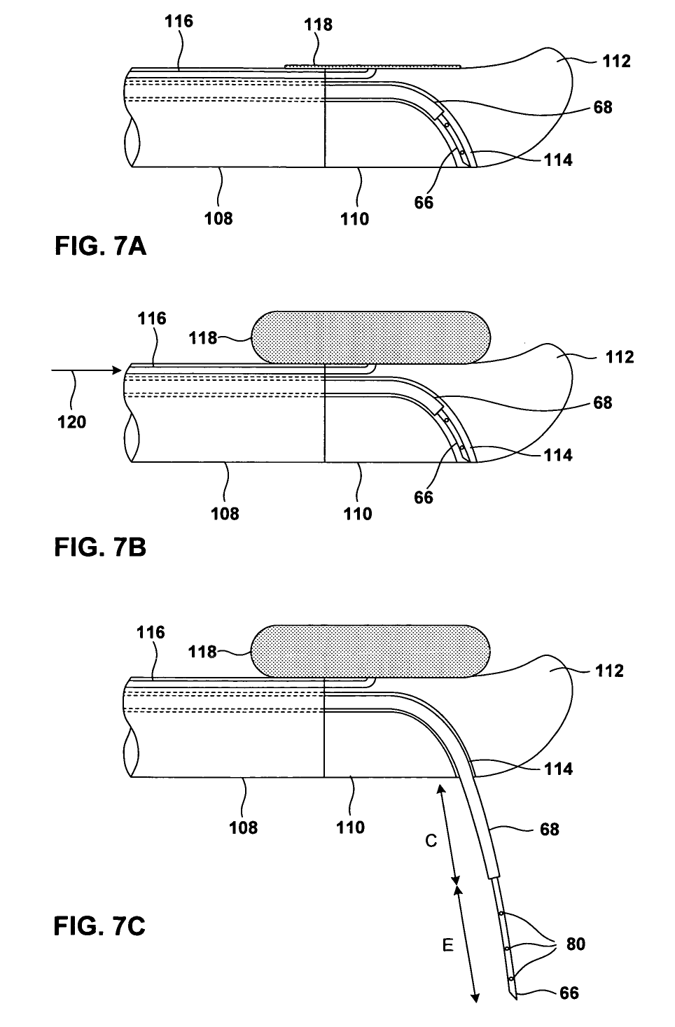

FIGS. 7A, 7B and 7C are cross-sectional side views of a distal end of and exemplary flexible catheter in which an inflatable balloon is used as a control mechanism. FIGS. 7A, 7B and 7C may be embodiments of flexible catheters 22, 44, 50 or 56, but flexible catheter 22 of FIG. 2 is referenced herein. As shown in FIG. 7A, shaft 108 is coupled to tip 110 at the distal end of catheter 22, where shaft 108 and tip 110 are similar to shaft 34 and tip 36. Tip 110 includes protrusion 112 that aids in catheter insertion through urethra 51. Tip 110 also includes channel 114 which allows needle 66 to exit tip 110. Needle 66 is insulated with sheath 68, such that the exposed portion of needle 66 may act as an electrode. A second needle (not shown) resides behind needle 66 and cannot be seen in FIG. 7A. Both needles may form an angle in the plane normal to the side view of FIG. 7A.

Shaft 108 and tip 110 also contain fluid channel 116 which terminates at the top of tip 110. Fluid channel 116 is in fluid communication with a pump located on generator 14 or another pressure vessel, i.e. a syringe. Inflatable balloon 118 is attached to fluid channel 116 to allow fluid from the fluid channel to enter the inflatable balloon. Preferably, inflatable balloon 118 is located on the side opposite the opening of channel 114. In this manner, inflatable balloon 118 may press against the wall of urethra 51 and subsequently force tip 110 against the wall of the urethra.

Inflatable balloon 118 may be constructed of a pliable polymer such as latex or polyvinylchloride (PVC) or a rigid polymer that is folded or creased when not inflated. In either case, the material should be capable of retaining small molecules such as nitrogen or oxygen. Inflatable balloon 118 may be capable of withstanding an internal pressure between 0 bar and 5 bar of pressure, where 0 bar is atmospheric pressure.

Channel 114 continues from tip 110 through shaft 108. The curved portion of channel 114 in tip 110 deflects needle 66 such that the needle penetrates the target tissue from the side of flexible catheter 22. The curvature of channel 114 may be altered to produce different entry angles of needle 66 and the second needle. However, the needles may not extend beyond the distal end of tip 110. In other words, the needles may exit at or near the side of flexible catheter 22, wherein the side is a lengthwise edge substantially facing the wall of urethra 51. The wall of urethra 51 is a tissue barrier as it surrounds flexible catheter 22. In some embodiments, the distal ends of needle 66 or the second needle may stop at a point further from the distal end of tip 110.

FIG. 7B shows inflatable balloon 118 being inflated with a fluid. The fluid flows through fluid channel 116 in the direction of arrow 120. The fluid may come from the same source as the conductive fluid or cooling fluid used for ablation therapy. Preferably, the fluid in fluid channel 116 is stored in a separate reservoir and includes separate control mechanisms. Inflatable balloon 118 expands from the pressure exerted by the fluid and presses against urethra 51. Inflatable balloon 118 may be between 2 mm and 20 mm in diameter when fully inflated and between approximately 4 mm and 50 mm in length. The length of inflatable balloon 118 may be parallel to the length of flexible catheter 22. The fluid may be either a liquid or a gas. Exemplary fluids are sterilized water, air, nitrogen, saline, alcohol, or any other fluid that may flow into inflatable balloon 118. Preferably, the fluid would not cause harm to patient 12 during a leak and is at or near room temperature. Pressure within inflatable balloon 118 may be monitored and kept constant with an automatic or manual pump.

In some embodiments, more than one inflatable balloon 118 may be included on flexible catheter 22. Each inflatable balloon may be inflated to different pressures, have different sizes, or be placed at varying locations along flexible catheter 22. Multiple inflatable balloons may allow flexible catheter 22 to accommodate complex passages or direct precise forces to certain areas of the flexible catheter. In other embodiments, inflatable balloon 118 may be used in conjunction with a pull-wire to provide the clinician with multiple mechanisms for successfully ablating prostate 24.

Alternatively, inflatable balloon 118 may have a bias shape to facilitate a specific placement. The shape of inflatable balloon 118 may be created by differing wall thicknesses, different materials, material dimensions, or multiple internal chambers. For example, inflatable balloon 118 may be shaped to be larger at the distal end than the proximal end. In this manner, more force is applied against the distal end of flexible catheter 22 than at the proximal end of the catheter.

As shown in FIG. 7C, needle 66 has been deployed from tip 110 of flexible catheter 22 after inflatable balloon 118 has been inflated. The exposed length E of needle 66 is variable by controlling the position of sheath 68. The covered length C of needle 66 is that length of the first needle outside of tip 110 that is not delivering energy to the surrounding tissue. Exposed length E may be controlled by the clinician to be generally between 1 mm and 50 mm. More specifically, exposed length E may be between 12 mm and 22 mm. Covered length C may be generally between 1 mm and 50 mm. Specifically, covered length C may also be between 12 mm and 22 mm. Once needle 66 and the second needle are deployed, the needles may be locked into place until the ablation therapy is completed. Needle 66 and the second needle are identical to those described in FIGS. 5A, 5B and 5C.

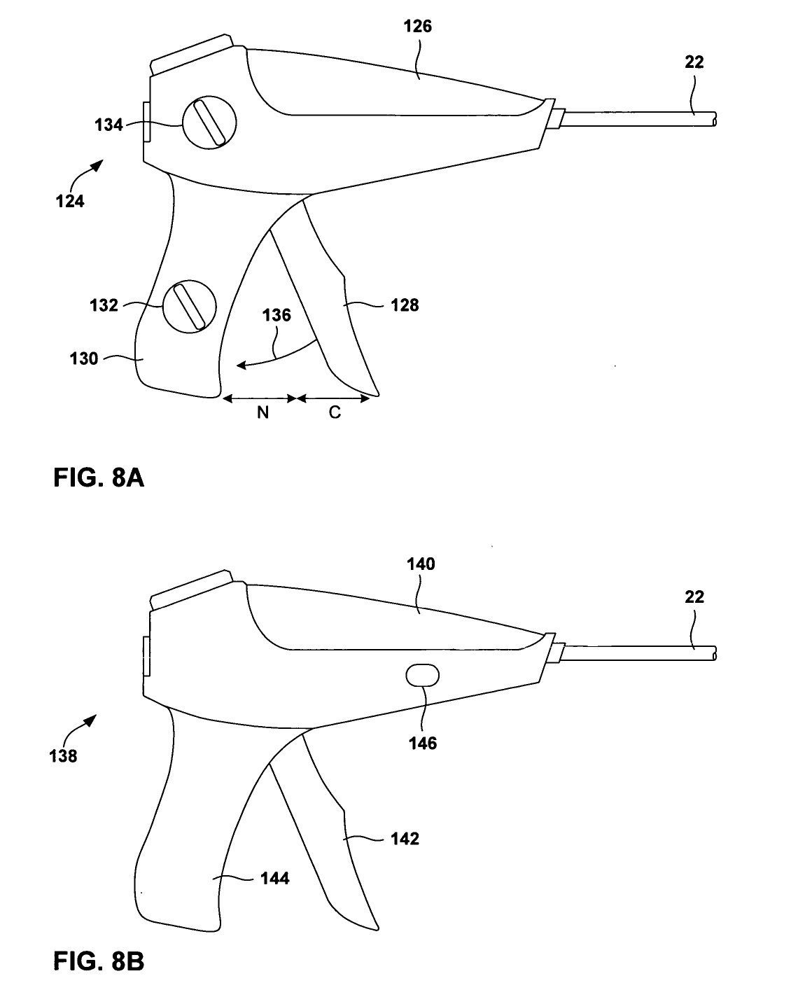

FIGS. 8A and 8B are exemplary hand pieces that provide control of an inflatable balloon. As shown in FIG. 8A, therapy device 124 includes housing 126, trigger 128 and handle 130 that may be alternative embodiments of housing 26, trigger 28 and handle 30 of therapy device 20 of FIG. 2. Flexible catheter 22 is inserted into housing 126, and trigger 128 and handle 130 are attached to housing 126. Dial 132 controls the length of needle 66 and any other needles while dial 134 controls the inflation of inflatable balloon 118.

Before flexible catheter 22 is inserted into urethra 51, the clinician rotates dials 90 and 92 to set the limits for each mechanism. For example, dial 132 may set the deployed length of needle 66 to any length between 12 mm and 22 mm and dial 134 may limit the pressure or flow rate of fluid delivered to inflatable balloon 118. The clinician may squeeze trigger 128 in the direction of arrow 136 the distance C to inflate inflatable balloon 118 and force tip 110 against the wall of urethra 51. The clinician may need to squeeze with greater force to continue moving trigger 128 the distance N. Distance N corresponds to deploying needle 66 into prostate 24. Dial 134 may include a ratcheting mechanism or locking mechanism to secure the pressure or flow rate setting.

In some embodiments, trigger 128 may not be used to inflate inflatable balloon 118. Instead, dial 134 may be used to directly control the flow of fluid into inflatable balloon 118. Dial 134 may specifically control the pressure within inflatable balloon 118 or the rate of fluid flow to the balloon. A pressure sensor may sense fluid pressure while a flow sensor may monitor the flow rate of the fluid. In some embodiments, therapy device 124 may include dials 132 and 134 located on any side of housing 126 or handle 130, or in other locations on therapy device 124. In other embodiments, multiple triggers 128 or dials 134 may each control separate inflatable balloons on flexible catheter 22. Alternatively, one dial may be used to selectively operate one or more inflatable balloons.

FIG. 8B, shows therapy device 138 including housing 140, trigger 142 and handle 144 that may be alternative embodiments of housing 26, trigger 28 and handle 30. Therapy device 138 may be an alternative of therapy device 20. Flexible catheter 22 is inserted into housing 140, and trigger 142 and handle 144 are attached to housing 140. Button 146 controls the delivery of fluid to inflatable balloon 118. The clinician presses button 146 to control either pressure or flow rate, which may be predetermined or clinician controlled. Preferably, a preset pressure limit is utilized such that the clinician can just press button 146 and inflatable balloon 118 is automatically inflated appropriately. Button 146 may be located at any other position on therapy device 138. For example, button 146 may be located on trigger 142.

Inflatable balloon 118 may be controlled through other mechanisms not located within a device similar to therapy device 20. For example, a simple syringe or manual pump may be used by the clinician to inflate the inflatable balloon. Other manual controls such as a hand pump or foot pump may also be employed.

In some embodiments, pull-wire 74 may be used in combination with inflatable balloon 118. Such a combination may provide multiple delivery options for the clinician in case of an abnormal procedure or patient 12 with other complications.

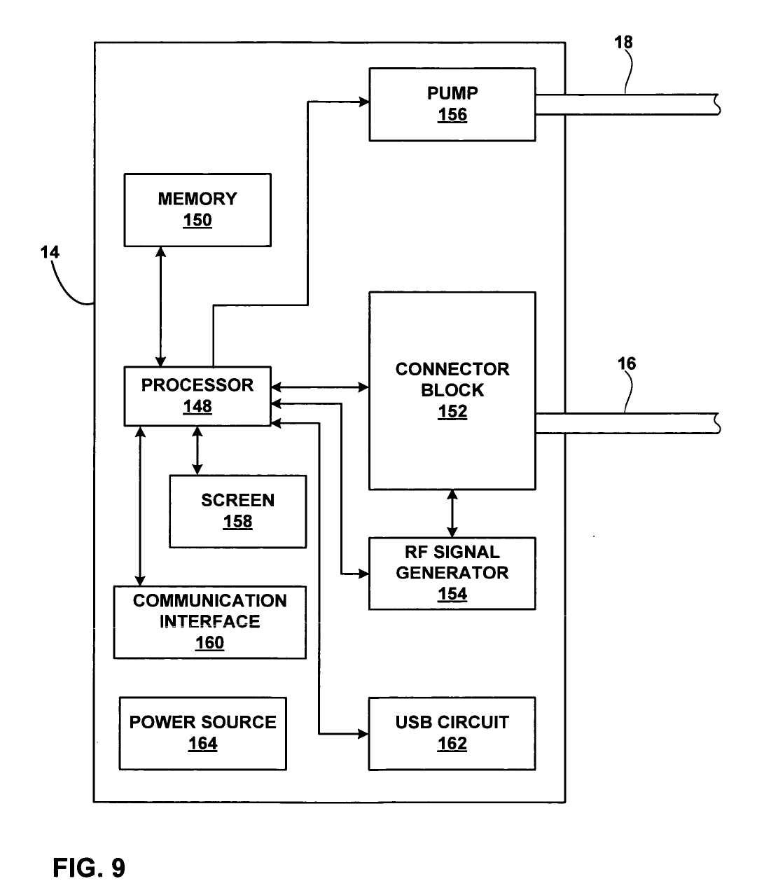

FIG. 9 is functional block diagram illustrating components of an exemplary generator system. In the example of FIG. 9, generator 14 includes a processor 148, memory 150, screen 158, connector block 152, RF signal generator 154, pump 156, communication interface 160, USB circuit 162, and power source 164. As shown in FIG. 9, connector block 152 is coupled to cable 16 for delivering RF energy produced by RF signal generator 154. Pump 156 produces pressure to deliver fluid through tube 18. The example of FIG. 9 may primarily used when flexible catheter 22 includes one or more pull-wires. However, an inflatable balloon may be used as a control mechanism if a separate fluid delivery device is used.

Processor 148 controls RF signal generator 154 to deliver RF energy therapy through connector block 152 according to therapy parameter values stored in memory 150. Processor 148 may receive such parameter values from screen 158 or communication interface 160 or USB circuit 162. When signaled by the clinician, which may be a signal from therapy device 20 conveyed through connector block 152, processor 148 communicates with RF signal generator 154 to produce the appropriate RF energy. As needed, pump 156 provides fluid to irrigate the ablation site or provides fluid to the electrode during wet electrode ablation. Fluid from pump 156 may also be diverted into inflatable balloon 118 if the balloon is utilized by therapy device 20.

In a preferred embodiment, the RF signal generator may have certain performance parameters. In this exemplary case, the generator may provide RF energy into two channels with a maximum of 50 Watts per channel. The ramp time for a 50 Watt change in power may occur in less than 25 milliseconds. The output power may be selected in 1 Watt steps. The maximum current to be provided to the patient may be 1.5 Amps, and the maximum voltage may be 180 Volts.

Connector block 152 may contain an interface for a plurality of connections, not just the connection for cable 16. These other connections may include one for a return electrode, a second RF energy channel, or separate sensors. As mentioned previously, connector block 152 may be a variety of blocks used to diagnose or treat a variety of diseases. All connector blocks may be exchanged and connect to processor 148 for proper operation. Pump 156 may be replaceable by the clinician to replace a dysfunctional pump or use another pump capable of pumping fluid at a different flow rate.

Processor 148 may also control data flow from the therapy. Data such as RF energy produced and fluid flow may be channeled into memory 150 for analysis. Processor 148 may comprise any one or more of a microprocessor, digital signal processor (DSP), application specific integrated circuit (ASIC), field-programmable gate array (FPGA), or other digital logic circuitry. Memory 150 may include multiple memories for storing a variety of data. For example, one memory may contain therapy parameters, one may contain generator operational files, and one may contain therapy data. Memory 150 may include any one or more of a random access memory (RAM), read-only memory (ROM), electronically-erasable programmable ROM (EEPROM), flash memory, or the like.

Processor 148 may also send data to USB circuit 162 when a USB device is present to save data from therapy. USB circuit 162 may control both USB ports in the present embodiment; however, USB circuit 162 may control any number of USB ports included in generator 14. In some embodiments, USB circuit may be an IEEE circuit when IEEE ports are used as a means for transferring data.

The USB circuit may control a variety of external devices. In some embodiments, a keyboard or mouse may be connected via a USB port for system control. In other embodiments, a printer may be attached via a USB port to create hard copies of patient data or summarize the therapy. Other types of connectivity may be available through the USB circuit 162, such as internet access.

Communications with generator 14 may be accomplished by radio frequency (RF) communication or local area network (LAN) with another computing device or network access point. This communication is possible through the use of communication interface 80. Communication interface 160 may be configured to conduct wireless or wired data transactions simultaneously as needed by the clinician.

Generator 14 may communicate with a variety of devices to enable appropriate operation. For example, generator 14 may utilize communication interface 160 to monitor inventory, order disposable parts for therapy from a vendor, and download upgraded software for a therapy. For example, generator 14 may order a new flexible catheter 22 if the catheter no longer operates correctly. In some embodiments, the clinician may communicate with a help-desk, either computer directed or human staffed, in real-time to solve operational problems quickly. These problems with generator 14 or a connected therapy device may be diagnosed remotely and remedied via a software patch in some cases.

Screen 158 is the interface between generator 14 and the clinician. Processor 148 controls the graphics displayed on screen 158 and identifies when the clinician presses on certain portions of the screen 158, which is sensitive to touch control. In this manner, screen 158 operation may be central to the operation of generator 14 and appropriate therapy or diagnosis.

Power source 164 delivers operating power to the components of generator 14. Power source 164 may utilize electricity from a standard 115 Volt electrical outlet or include a battery and a power generation circuit to produce the operating power. In some embodiments, the battery may be rechargeable to allow extended operation. Recharging may be accomplished through the 115 Volt electrical outlet. In other embodiments, traditional batteries may be used.

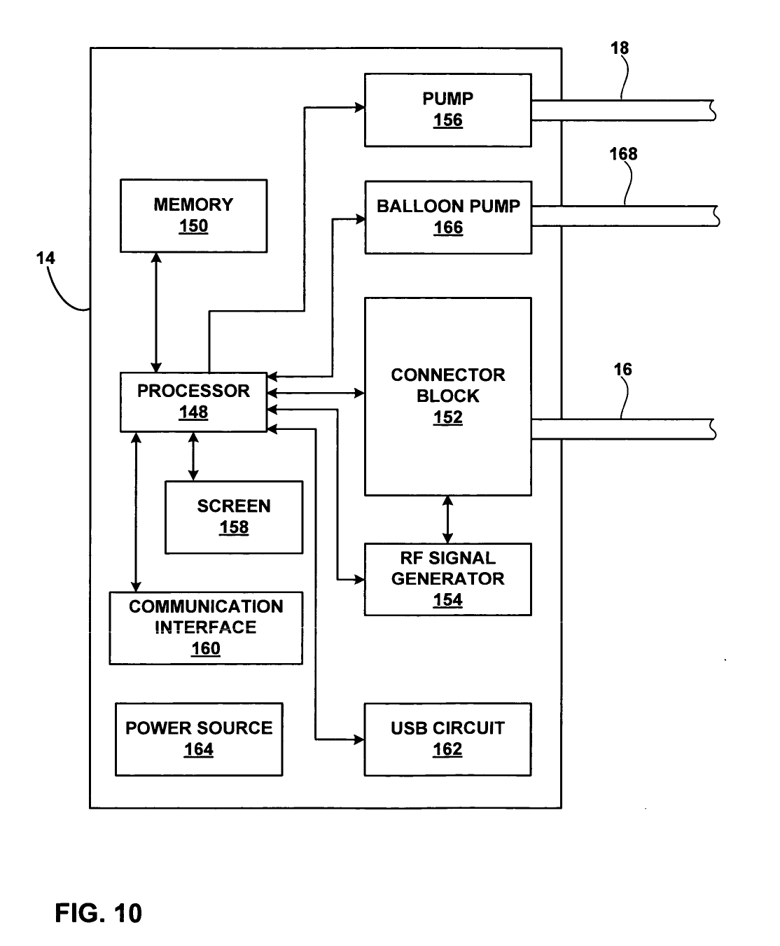

FIG. 10 is functional block diagram illustrating components of an exemplary generator system that controls an inflatable balloon. The example of FIG. 10 is substantially similar to FIG. 9. However, FIG. 10 shows components of a generator 14 that also includes balloon pump 166 for delivering fluid to inflatable balloon 118 at the distal end of flexible catheter 22. Balloon pump 166 may be a peristaltic pump or controlled piston capable of producing flow rates between 0.01 milliliters (mL) and 100 mL per minute. Fluid is pumped from balloon pump 166, through fluid line 168, and into therapy device 20. Fluid line 168 may be independent or located within tube 18. The source of fluid for balloon pump 166 may be similar or different than the source for pump 156.

A pressure or flow sensor (not shown) may be located at the exit of balloon pump 166 or near inflatable balloon 118. The sensor provides feedback control for balloon pump 166. Balloon pump deflates inflatable balloon 118 to control balloon pressure or before removing flexible catheter 22 from urethra 51. When utilizing the embodiment of therapy device 138, button 146 may be electrically coupled to generator 14, where processor 148 controls balloon pump 166 according to clinician input. Other therapy devices may include user inputs as well.

Processor 148 controls balloon pump 166 operation based upon user parameters selected on screen 158 and instructions contained in memory 150. Processor 148 may monitor any sensors related to the fluid flow to control the inflation of inflatable balloon 118 and save some or all of the measured data in memory 150. In other embodiments, balloon pump 166 may contain independent processing and memory circuitry such that the pump may operate at modularly.

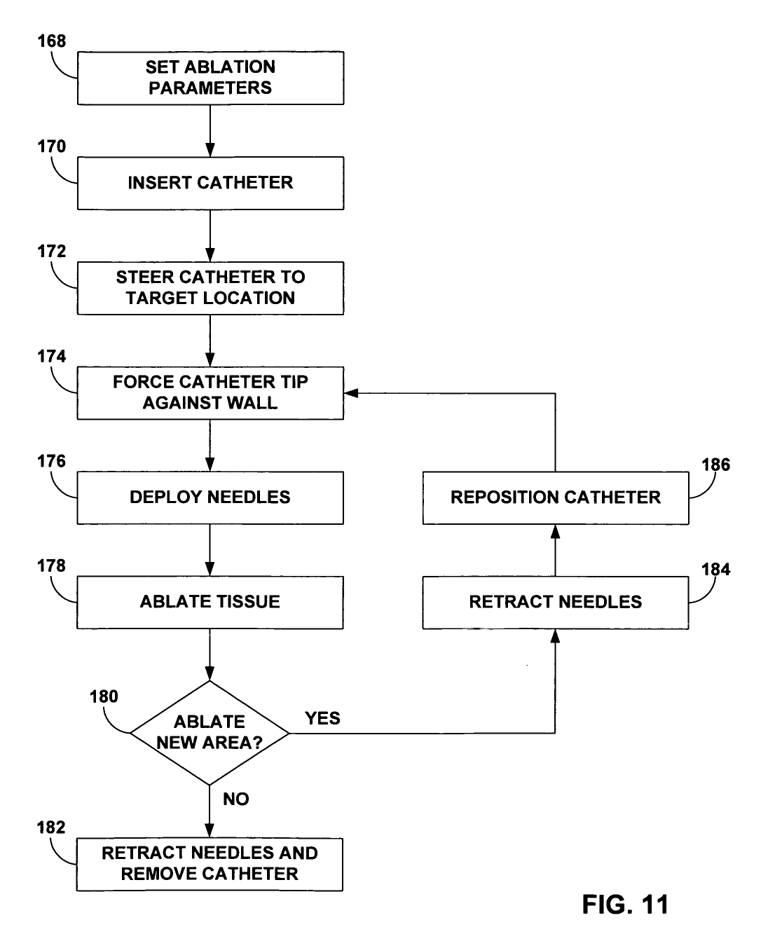

FIG. 11 is a flow diagram illustrating an example technique for positioning a flexible catheter with a pull-wire and ablating tissue. As shown in FIG. 11, pull-wire 74 is used as the control mechanism of flexible catheter 22. The clinician sets ablation parameters in generator 14 (168). Ablation parameters may include RF power, needle lengths, pull-wire limits, or other parameters related to the therapy. Selecting a desired flexible catheter 22 configuration may be an ablation parameter as well. The clinician next inserts flexible catheter 22 into urethra 51 of patient 12 (170) and steers the catheter with pull-wire 74 to correctly place tip 36 adjacent to prostate 24 (172). The clinician may use a flexible cystoscope within flexible catheter 22 to guide the catheter. In some embodiments, pull-wire 74 is not used to steer flexible catheter 22. Once correctly positioned, the clinician tightens pull-wire 74 to force a side of flexible catheter 22 against the wall of urethra 51 (174). The clinician then deploys needle 66 and any other needles into prostate 24 at the forced area of the urethra wall (176).

The clinician starts tissue ablation by pressing a button on generator 14 or therapy device 20 (178). Conductive fluid may or may not be delivered by the needles. If the clinician does not want to ablate a new area of prostate 24 (180), the clinician retracts the needles, releases pull-wire 74, and removes flexible catheter 22 from patient 12 (182). If the clinician desires to ablate more tissue, the clinician retracts the needles and releases the pull-wire 74 (184), repositions flexible catheter 22 adjacent to the new tissue area (186), and tightens pull-wire 74 to again force a side of flexible catheter 22 against the wall of urethra 51 (174). Needles are again deployed (176) and ablation may begin again to treat more tissue (178).

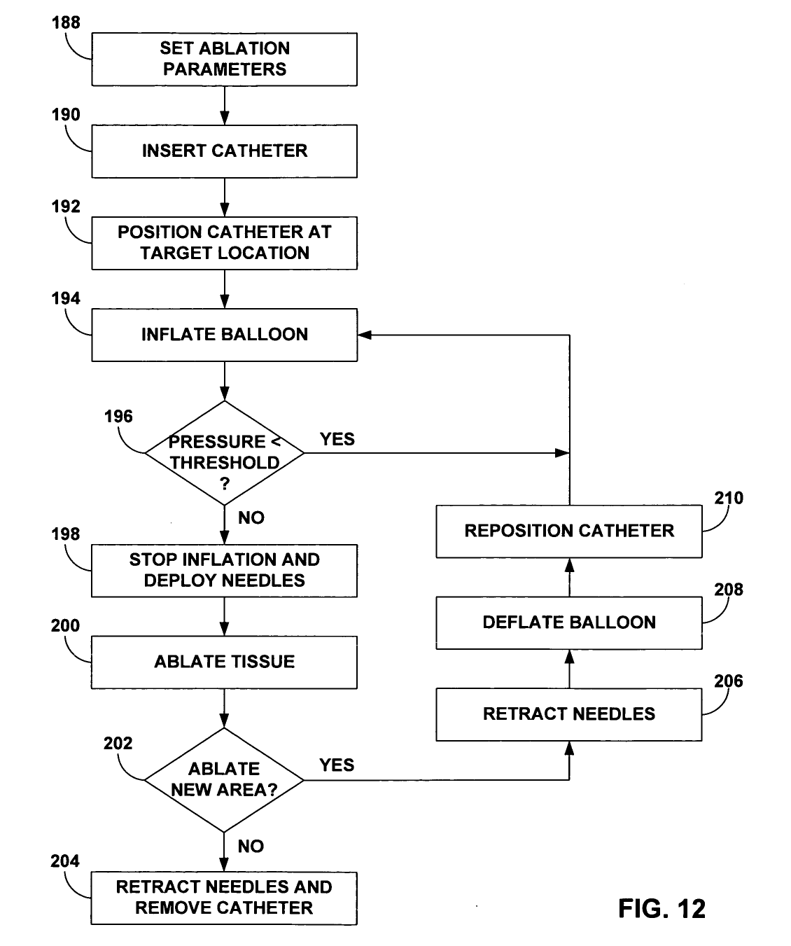

FIG. 12 is a flow diagram illustrating an example technique for positioning a flexible catheter with an inflatable balloon and ablating tissue. As shown in FIG. 12, inflatable balloon 118 is used as the control mechanism of flexible catheter 22. The clinician sets ablation parameters in generator 14 (188). Ablation parameters may include RF power, needle lengths, fluid pressure or flow limits for inflating balloon 118, or other parameters related to the therapy. Selecting a desired flexible catheter 22 configuration may be an ablation parameter as well. The clinician next inserts flexible catheter 22 into urethra 51 of patient 12 (190) and positions the catheter to correctly place tip 36 adjacent to prostate 24 (192). The clinician may use a flexible cystoscope within flexible catheter 22 to guide the catheter.

Once correctly positioned, the clinician begins inflating inflatable balloon 118 to force a side of flexible catheter 22 against the wall of urethra 51 (194). The clinician or generator 14 monitors the fluid pressure and compares it to a predetermined threshold (196). If the pressure is less than the threshold, the balloon is continued to be inflated (194). If the pressure is greater than the threshold, the clinician or generator stops inflation and deploys needle 66 and any other needles into prostate 24 at the forced area of the urethra wall (198).

The clinician starts tissue ablation by pressing a button on generator 14 or therapy device 20 (200). Conductive fluid may or may not be delivered by the needles. If the clinician does not want to ablate a new area of prostate 24 (202), the clinician retracts the needles, deflates inflatable balloon 118, and removes flexible catheter 22 from patient 12 (204). If the clinician desires to ablate more tissue, the clinician retracts the needles (206), deflates inflatable balloon 118 (208), and repositions flexible catheter 22 adjacent to the new tissue area (210). The clinician or generator 14 then re-inflates inflatable balloon 118 (194) and continues with further ablation therapy.

In some embodiments, inflatable balloon 118 may be inflated during flexible catheter 22 insertion to aid in catheter placement. Inflatable balloon 118 may aid in bending flexible catheter 22 around sharp angles or to dilate urethra 51 if it is too narrow. Alternatively, other inflatable balloons located along flexible catheter 22 may aid in catheter navigation instead.

Various embodiments of the described invention may include processors that are realized by microprocessors, Application-Specific Integrated Circuits (ASIC), Field-Programmable Gate Arrays (FPGA), or other equivalent integrated logic circuitry. The processor may also utilize several different types of storage methods to hold computer-readable instructions for the device operation and data storage. These memory and storage media types may include a type of hard disk, random access memory (RAM), or flash memory, e.g. CompactFlash or SmartMedia. Each storage option may be chosen depending on the embodiment of the invention. Generator 14 may contain permanent memory or a more portable removable memory type to enable easy data transfer for offline data analysis.

The preceding specific embodiments are illustrative of the practice of the invention. It is to be understood, therefore, that other expedients known to those skilled in the art or disclosed herein may be employed without departing from the invention or the scope of the claims.

Many embodiments of the invention have been described. Various modifications may be made without departing from the scope of the claims. These and other embodiments are within the scope of the following claims.

1. A method for ablating tissue, the method comprising:
inserting a flexible catheter into a passage;
biasing a first side of the flexible catheter against a wall of the passage;
extending a needle from the first side of the flexible catheter through the wall of the passage and into a target tissue; and
delivering energy via the needle to ablate at least a portion of the target tissue.

2. The method of claim 1, wherein the first side of the flexible catheter is near a distal end of the flexible catheter.

3. The method of claim 1, wherein forcing the first side of the flexible catheter against the wall of the passage is performed by pulling a pull-wire located within the flexible catheter.

4. The method of claim 3, further comprising steering the flexible catheter through the passage.

5. The method of claim 3, further comprising locking the pull-wire in place once the first side of the flexible catheter is against the wall of the passage.

6. The method of claim 3, wherein two or more pull-wires are located within the flexible catheter.

7. The method of claim 1, wherein forcing the first side of the flexible catheter against the wall of the passage is performed by inflating a balloon attached to a second side of the flexible catheter.

8. The method of claim 7, further comprising pumping a fluid through the flexible catheter to inflate the balloon.

9. The method of claim 1, further comprising rotating the flexible catheter to position the flexible catheter within the passage.

10. The method of claim 1, further comprising delivering a conductive fluid to the target tissue via the needle.

11. The method of claim 10, further comprising moving the conductive fluid through a plurality of holes in the needle.

12. The method of claim 1, further comprising inserting a flexible cystoscope into the flexible catheter.

13. The method of claim 1, wherein the passage includes the urethra.

14. The method of claim 1, wherein the target tissue is the prostate.

15. A system for ablating tissue, the system comprising:
a generator that generates energy to ablate at least a portion of a target tissue;
a flexible catheter that is inserted into a passage;
a housing that accepts the flexible catheter;
a control mechanism that biases a first side of the flexible catheter against a wall of the passage; and
a needle that extends from the first side of the flexible catheter through the wall of the passage and into the target tissue to deliver the energy.

16. The system of claim 15, wherein the first side of the flexible catheter is near a distal end of the flexible catheter.

17. The system of claim 15, wherein the control mechanism is a pull-wire located within the flexible catheter.

18. The system of claim 17, further comprising a ratcheting mechanism on the housing, wherein the ratcheting mechanism is attached to the pull-wire and pulls the wire towards the housing.

19. The system of claim 17, wherein the control mechanism is two or more pull-wires located at different circumferential positions within the flexible catheter.

20. The system of claim 15, wherein the control mechanism is a balloon attached to a second side of the flexible catheter.

21. The system of claim 20, further comprising a pump that pumps a fluid through the flexible catheter and into the balloon, wherein the fluid causes the balloon to expand.

22. The system of claim 15, wherein at least a portion of the flexible catheter is rigid.

23. The system of claim 15, further comprising a pump that delivers a conductive fluid through the needle, wherein the needle comprises a plurality of holes that allow the conductive fluid to enter the target tissue.

24. The system of claim 15, further comprising a flexible cystoscope that is insertable into the flexible catheter.

25. The system of claim 15, wherein the passage includes the urethra.

26. The system of claim 15, wherein the target tissue is the prostate.

27. A device for accessing a tissue to be ablated, the device comprising:
a flexible catheter that is inserted into a passage;
a control mechanism that biases a first side of the flexible catheter against a wall of the passage;
a needle that extends from the first side of the flexible catheter through the wall of the passage and into a target tissue, wherein the first side of the flexible catheter is near a distal end of the flexible catheter; and
an axial channel that accepts a flexible cystoscope.

28. The device of claim 27, wherein the control mechanism is a pull-wire located within the flexible catheter.

29. The device of claim 27, wherein the control mechanism is a balloon attached to a second side of the flexible catheter.

30. The device of claim 27, wherein at least a portion of the flexible catheter is rigid.

31. The system of claim 27, wherein the flexible catheter is bent to a radius of curvature greater than 2 cm.

32. The device of claim 27, wherein the flexible catheter is flexible within one plane.

33. The device of claim 27, wherein the flexible catheter may bend a plurality of locations along the length of the flexible catheter.

34. The device of claim 27, wherein the passage is the urethra.
